(19) United States
(12) Patent Application Publication
(10) Pub. No.: US 2010/0047371 A1
(43) Pub. Date: Feb. 25, 2010

(54) COMPOSITIONS AND METHODS FOR ORAL CANCER CHEMOPREVENTION USING BERRY PREPARATIONS AND EXTRACTS

(76) Inventors: Russell J. Mumper, Chapel Hill, NC (US); Susan R. Mallery, Columbus, OH (US); Gary D. Stoner, Worthington, OH (US); Peter E. Larsen, Powell, OH (US)

Correspondence Address: KING & SCHICKLI, PLLC, 247 NORTH BROADWAY, LEXINGTON, KY, 40507, US

(21) Appl. No.: 12/439,288

(22) PCT Filed: Aug. 29, 2006
(86) PCT No.: PCT/US06/33735

§ 371 Date: Oct. 1, 2009

(51) Int. Cl. A61K 36/73; A61K 36/00; A61P 35/00

(52) U.S. Cl. 424/765; 424/777

(57) ABSTRACT

A composition is provided for chemoprevention of an oral cancer or precancerous condition, including an isolated berry preparation admixed with a bioadhesive carrier. The carrier may be a mucoadhesive gel. The berry preparation may be derived from one or more of strawberry, raspberry, red raspberry, and black raspberry. Methods for producing the compositions are provided also. There are also provided methods for chemoprevention of an oral cancer or precancerous condition, utilizing the compositions of the invention. Still further, methods for enhancing stability of an anthocyanin contained in the berry preparation of the invention are provided, along with methods for enhancing efficacy of the compositions.

This invention was made with U.S. Government support under NIH Grant Number NIH-NCI R21CA11120 and USDA Grant Number 2003-34501-13965. The U.S. Government may have certain rights in this invention.

### TECHNICAL FIELD

The present invention relates to compositions for chemoprevention of oral cancer and precancerous lesions, and for methods for preparing the compositions. Specifically, the invention relates to bioadhesive gels containing an isolated berry extract, formulated for local delivery for the chemoprevention of oral cancer and precancerous lesions. The invention relates also to methods for stabilizing and enhancing the efficacy of chemopreventive components of the compositions.

### BACKGROUND OF THE INVENTION

Oropharyngeal cancer annually affects approximately 29,370 persons in the U.S., and more than 7,200 Americans die each year from this disease [Jemal et al., 2005]. Despite concerted efforts to improve treatments, five year survival rates for patients with advanced stage oral squamous cell carcinoma (oral SCC) remain among the lowest of solid cancers. For those persons who are cured, morbidity remains high due to loss of tissues that are critical for aesthetics and function [Malone et al., 2004]. Another significant concern for persons diagnosed with oral SCC is the potential for tumor recurrence or the development of a second primary cancer [Braakhuis et al., 2002; Pathak et al., 2005; Schwartz et al., 2000]. Clearly, early detection of precancerous oral lesions combined with strategies for local intervention to prevent progression to overt oral SCC could dramatically improve clinical outcomes.

Cancer chemoprevention, which represents one promising approach, is defined as the prevention, inhibition or reversal of carcinogenesis by intervention with chemically derived or naturally occurring dietary substances. While this concept is conceptually appealing, the results of previously conducted oral cavity chemoprevention trials have not been promising. Although pharyngeal premalignant lesions often respond to chemoprevention therapy, oral cavity lesions are frequently nonresponsive, even to combination agents such as cis-retinoic acid, IFN-α, and α-tocopherol cocktail [Toma et al., 2004]. Furthermore, although some agents such as vitamin A derivatives were partially effective in managing oral premalignant lesions, these formulations often caused undesirable toxicities such as severe mucositis [Papadimitrakopoulou et al., 1999]. Notably, the majority of previously conducted oral chemoprevention trials employed systemic agent administration. Local delivery methods, however, are recognized as having advantages for site-specific, targeted disease management. Local therapies provide high, clinically effective concentrations directly to the treatment site, without causing deleterious systemic side effects. Local applications are not always a panacea; however, as it is necessary to carefully select diseases amenable to such treatments. Further, for persons with oral cavity lesions such as those encountered in oral epithelial dysplasia, chemoprevention is likely to be necessary for the remainder of their lives.

Consequently, chemopreventive compounds selected for treatment and/or prevention of recurrence of cancerous and pre-cancerous conditions must be both effective and non-toxic for long term use. Recent studies from the present inventors have demonstrated that berries such as black raspberries possess cancer-preventing properties at both in-vitro and in-vivo levels [Kresty et al., 2001; Castro et al., 2002; Xue et al., 2001; Huang et al., 2002; Rodrigo et al, in press; U.S. patent application Ser. No. 10/951,413]. In particular, the compounds which provide natural colors to fruits and vegetables i.e., anthocyanins, have been shown to demonstrate a wealth of chemopreventive properties [Hecht et al., in press; Liu et al., 2002; Katsube et al., 2003; Hu et al., 2003]. Furthermore, berries such as black raspberries also contain additional known chemopreventive compounds including other polyphenols such as ellagic acid, ferulic acid, coumaric acid, quercetin, and others, vitamins including A, C, E, folic acid, and others, minerals such as calcium and selenium, and multiple phtyosterols such as beta-sitosterol, kaempasterol, and others. In addition, the present inventors have confirmed that dietary administration of high dosages of freeze-dried black raspberries for extended periods of time is well tolerated [Stoner et al., 2005]. Finally, when contemplating development of a natural product based treatment, it is beneficial to first identify its bioactive constituents as preservation of these agents indicates compound stability in the final formulation. Notably, recent characterization studies by Hecht et al., which used spectral properties as well as comparison with anthocyanin standards, identified cyanidin glycosides as bioactive compounds in black raspberry extracts [Hecht et al., in press].

The present invention addresses the identified need in the art by providing compositions for chemoprevention of oral cancer and precancerous conditions, and methods for making such compositions. Advantageously, the compositions of the present invention are stable and formulated for local delivery, thereby maximizing efficacy. The compositions provide stability for the chemopreventive agents, and further provide good absorptive capacity.

### SUMMARY OF THE INVENTION

In accordance with the foregoing need identified in the art, the present invention provides compositions for chemoprevention of oral cancerous and precancerous conditions. In one aspect, the present invention provides a composition for chemoprevention of an oral cancer, comprising an isolated berry preparation admixed with a bioadhesive carrier. The isolated berry preparation comprises at least one anthocyanin in an amount effective for chemoprevention of the oral cancer, and may be derived from one or more of strawberry, raspberry, red raspberry, black raspberry, and combinations thereof. In one embodiment of the invention, the isolated berry preparation is derived from black raspberry. Typically, the berry preparation will be prepared by physically disrupting and concentrating a suitable quantity of berries, such as by freeze-drying, solvent extraction, or combinations of the methods. In one embodiment, a freeze-dried berry preparation may be prepared by the process of freeze-drying or lyophilizing the berry preparation to produce a dry powder up to 10% weight by weight residual water. In another embodiment, a solvent-extracted berry preparation may be prepared by exposing the freeze-dried preparation to a suitable solvent such as ethanol, methanol, acetone, chloroform, methylene chloride, or mixtures thereof with water to enrich the bioactive components contained in the preparation. In still yet another embodiment, a solvent-extracted berry preparation may be prepared by exposing the physically disrupted berries to a suitable solvent such as ethanol, methanol, acetone, chloroform, methylene chloride, or mixtures thereof with water to enrich the bioactive components contained in the preparation. The freeze-dried berry preparation and the solvent-extracted berry preparation are hereinafter referred to as an isolated berry preparation. Typically, for use in formulations for human or animal treatment, suitable approved solvents will be used, such as for example ethanol.

For storage, the isolated berry preparation admixed with a bioadhesive carrier may be adjusted to a pH of from about 3.0 to about 7.0. In one embodiment, the isolated berry preparation admixed with a bioadhesive carrier is adjusted to a pH of from about 3.0 to about 4.5 for storage. In another embodiment, the isolated berry preparation admixed with a bioadhesive carrier is adjusted to a pH of about 3.5 for storage. The composition of the present invention may include from about 1% (w/w) to about 10% (w/w) concentrated berry preparation. In certain embodiments, the composition may include from about 5% (w/w) to about 10% (w/w) concentrated berry preparation.

In one embodiment of the present composition, the bioadhesive carrier is a mucoadhesive gel adapted for transmucosal delivery of the isolated berry preparation. The skilled artisan will appreciate that this will desirably allow penetration of chemopreventive compounds into the epithelial mucosal tissue at a predetermined site for treatment. The mucoadhesive gel carrier may be provided at a final viscosity of from about 5,000 cP to about 30,000 cP. It will be appreciated by the skilled artisan that the carrier should be viscous enough to allow it to remain on the tissues for a suitable amount of time to achieve a desired a biological effect, but not so viscous as to prevent its ability to be spread over the tissue. In one embodiment, the carrier is provided at a final viscosity of from about 10,000 cP to about 20,000 cP. It will be appreciated by the skilled artisan that the term “mucoadhesive” refers in general to the ability of a material to adhere to a mucous membrane, to allow adhesion of the material to the tissue for an extended period of time. Mucous membranes may include a variety of tissues, including, but not limited to, ophthalmic, oropharyngeal, sublingual, gingival, buccal, nasal, intestinal, vaginal, and rectal tissues.

The skilled artisan will further appreciate that this property is desirable for delivery of an active agent to a tissue site, such as for example transmucosal delivery. A number of bioadhesive carriers and mucoadhesive gels are known in the art, such as for example polyacrylic acid, cross-linked polyacrylic acid, hydroxypropylcellulose, hydroxyethylcellulose, polymethylmethacrylate, alginate, carboxymethylcellulose, polyvinylpyrrolidone, polyvinylalcohol, chitosan, gelatin, derivatives thereof, and combinations thereof. The skilled artisan will further appreciate that this list is by no means limiting, as numerous additional synthetic and natural polymers are known in the art to possess bioadhesive/mucoadhesive properties. In one embodiment, the present invention contemplates use of a mucoadhesive gel comprising high molecular weight polymers of acrylic acid crosslinked with polyalkyl ethers or divinyl glycol, provided under the trade names CARBOPOL and NOVEON by Noveon, Inc. (Cleveland, Ohio). However, the use of any suitable bioadhesive carrier as described above is contemplated for the present composition, in accordance with the intended use of the composition.

In another aspect, the present invention provides a method for chemoprevention of an oral cancer or precancerous condition, comprising topically administering to an individual in need of such chemoprevention an isolated berry preparation admixed with a bioadhesive carrier. The method contemplates administration of the composition to the interior of the oral cavity of the individual. In one embodiment, the isolated berry preparation is substantially as described above, being derived from one or more of strawberry, raspberry, red raspberry, black raspberry, and combinations, and comprising at least one anthocyanin in an amount effective for chemoprevention of the oral cancer. The berry preparation is disrupted, concentrated, and stored substantially as described above, i.e., by at least one of freeze-drying and solvent extraction.

The isolated berry preparation may be admixed with a suitable bioadhesive carrier to include from about 1% (w/w) to about 10% (w/w) concentrated berry preparation. In one embodiment, the berry preparation is admixed with the bioadhesive carrier to include from about 5% (w/w) to about 10% (w/w) concentrated berry preparation. The bioadhesive carrier may be a mucoadhesive gel as described above.

In yet another aspect, the present invention provides a method for stabilizing an anthocyanin contained in an isolated berry preparation, comprising the steps of admixing the isolated berry preparation with a bioadhesive carrier to form a mixture and adjusting the mixture to a pH of from about 3.0 to about 4.5. The bioadhesive carrier may be a mucoadhesive gel. In one embodiment, the pH of the mixture is adjusted to about 3.5. As described above, the isolated berry preparation may be derived from one or more of strawberry, raspberry, red raspberry, and black raspberry. Preparation of the isolated berry preparation is substantially as described above.

In still yet another aspect of the present invention, a method for preparing a composition for chemoprevention of an oral cancer or precancerous condition is provided, comprising the steps of physically disrupting a quantity of berries, concentrating the physically disrupted berries, and admixing the berry concentrate with a bioadhesive carrier. As described above, the physically disrupted berries may be concentrated by at least one of freeze-drying or solvent extraction such as, for example, water-ethanol extraction, to enrich the bioactive components contained in the preparation. In one embodiment, the physically disrupted berries may be concentrated by water ethanol extraction. For storage, the berry concentrate admixed with bioadhesive carrier may be adjusted to a pH of from about 3.0 to about 7.0. In one embodiment, the isolated berry preparation admixed with a bioadhesive carrier is adjusted to a pH of from about 3.0 to about 4.5 for storage. In another embodiment, the isolated berry preparation admixed with a bioadhesive carrier is adjusted to a pH of about 3.5 for storage. The isolated berry preparation may be derived from one or more of strawberry, raspberry, red raspberry, and black raspberry as is described above, and the composition may comprise from about 1% (w/w) to about 10% (w/w) concentrated berry preparation admixed with the bioadhesive carrier. In one embodiment, the composition contains from about 5% (w/w) to about 10% (w/w) concentrated berry preparation. The bioadhesive carrier is substantially as described above.

Still yet further, the present invention provides a method for increasing the concentration of an anthocyanin in a bodily tissue or fluid, comprising applying an isolated berry preparation substantially as described above to an interior of an oral cavity of an individual at risk of an oral cancer. The bodily tissue bodily tissue or fluid contemplated may comprise a mucosal tissue, an oral mucosa tissue, oral tissue, peripheral blood, serum, and saliva. The pH of the described composition may be adjusted to from about 6.0 to about 7.0 prior to applying the gel to the oral cavity of the individual. In one embodiment of the method, the pH is adjusted to about 6.5 prior to applying.

In yet still another aspect of the present invention, a method is provided for improving the efficacy of a composition for chemoprevention of an oral cancer or precancerous condition, comprising the steps of providing an isolated berry preparation, storing the isolated berry preparation at a first pH, admixing the isolated berry preparation with a bioadhesive carrier to form a mixture, and adjusting the mixture to a second pH which is different from the first pH prior to applying the mixture to an oral cavity of an individual in need of chemoprevention. Typically, the second pH is greater than the first pH. The first pH may be from about 3.0 to about 4.5 and the second pH is from about 6.0 to about 7.0. In one embodiment of the invention, the first pH is about 3.5 and the second pH is about 6.5.

As has been previously described, the isolated berry preparation may be derived from one or more of strawberry, raspberry, red raspberry, and black raspberry. In one embodiment the isolated berry preparation is derived from black raspberry by the steps of physically disrupting a quantity of berries, followed by concentrating the physically disrupted berries by at least one of freeze-drying or solvent extraction. From about 1% (w/w) to about 10% (w/w) concentrated berry preparation may be admixed with the bioadhesive carrier. In one embodiment, from about 5% (w/w) to about 10% (w/w) concentrated berry preparation is admixed with the bioadhesive carrier. The bioadhesive carrier may be a mucoadhesive gel as described above.

As should be appreciated, the embodiments shown and described herein are an illustration of one of the modes best suited to carry out the invention. It will be realized that the invention is capable of other different embodiments and its several details are capable of modification in various, obvious aspects all without departing from the invention. Accordingly, the drawings and descriptions will be regarded as illustrative in nature, and not as restrictive.

### BRIEF DESCRIPTION OF THE DRAWINGS

The accompanying drawings incorporated in and forming a part of the specification, illustrate several aspects of the present invention and together with the description serve to explain the principles of the invention. In the drawings:

FIG. 1 shows a standard HPLC chromatogram of anthocyanins in freeze-dried black raspberries, with FBR 1 corresponding to cyanidin 3-glucoside and cyanidin 3-sambubioside and FBR 2 corresponding to cyanidin 3-xylosylrutinoside and cyanidin 3-rutinoside;

FIG. 2 depicts a standard curve for FBR 1 and FBR 2 anthocyanin peaks in freeze-dried black raspberries. Both FBR 1 and FBR 2 were linear in the concentration range of 50 to 2000 μg/mL;

FIGS. 3A and 3B show anthocyanin stability in the berry gels as measured by retention of FBR 1 and FBR; FIG. 3A: Anthocyanin stability in the berry gels at 7 days as a function of final gel pH (pH 4.5, 5.5, 6.5, and 7.5) and storage temperature (4, 25, and 40°); FIG. 3B: Anthocyanin stability in the berry gels over 28 days as a function of final gel pH (pH 3.5 and 4.0); and

FIG. 4 shows a representative mass spectroscopy HPLC chromatogram of an oral mucosal tissue explant following topical application of berry gel containing 10% w/w FBR, showing the presence of peaks corresponding to the four anthocyanins present in black raspberries.

Reference will now be made in detail to the present preferred embodiment of the invention, an example of which is illustrated in the accompanying drawings.

### DETAILED DESCRIPTION OF THE INVENTION

The examples provided herein are presented in support of and to further illustrate the invention as described above, but are not to be considered as limited thereto. All references cited to herein are incorporated into the present disclosure in their entirety by reference unless otherwise specified. The following general of materials and methods applies to the examples set forth hereinafter.

Noveon AA1 (NF) was obtained from Noveon, Inc. (Cleveland, Ohio). Carbopol 971P NF was a gift from BF Goodrich Specialty Chemicals (Cleveland, Ohio). Edetate disodium (EDTA; USP), 2-phenoxyethanol (BP), benzyl alcohol (USP), glycerin (USP), sodium hydroxide (NF), and formic acid (ACS reagent) were obtained from Spectrum Quality Products, Inc. (New Brunswick, N.J.). Acetonitrile (HPLC grade) was from Fisher Scientific (Hampton, N.H.). Reagents used for tissue explant analyses (sodium carboxymethylcellulose, sorbitol, NaCl, KCl, calcium chloride dehydrate, MgCl₂, Trizma hydrochloride) were purchased from Sigma Chemical Company (St. Louis, Mo.).

All black raspberries used in these studies were of the Jewel variety (Rubus occidentalis) and were grown at the Stokes Raspberry Farm (Wilmington, Ohio). All berries were grown in the same part of the field, and picked at about the same degree of ripeness (when the majority of the berries in a cluster have turned black-this occurs within a period of 1-2 weeks). The berries were harvested mechanically (with a picker) in a total period of 4 hr, washed, and frozen at −20° C. within an hour of the time of harvest. The frozen berries were subsequently freeze-dried and ground into a powder as described previously [Kresty et al., 2001; Castro et al., 2002; Xue et al., 2001; Huang et al., 2002; Rodrigo et al., in press; Stoner et al., 2005].

All berries picked during a single year were freeze-dried concurrently. The freeze-drying process (including the entire black raspberry fruit including seeds) resulted in very little degradation of the analyzed components of the berries (see description below). The freeze-dried berry preparations (hereinafter, FBR) were stored frozen until use. Random samples of each batch of freeze-dried berries were shipped to an independent laboratory for measurement of the components to ensure that measured components did not degrade significantly during processing and storage as described above. On average, over a five year period, the variation in content of most measured components was less than 20%.

Anthocyanin content in sera, saliva, and tissue explants was evaluated by an HPLC-MS assay. The respective samples (sera, saliva or tissue homogenates) were dissolved in 90% solvent A (water plus 1% formic acid) and 10% solvent B (acetonitrile plus 1% formic acid) and then separated on a Symmetry C₁₈ reversed-phase column and analyzed using a Waters 2695 gradient HPLC separation module equipped with a 996 photodiode-array (PDA) UV/visible absorbance detector (Milford, Mass.). The solvent system consisted of a step gradient from 90% A to 50% B over 15 minutes, and the absorption spectra were recorded from 200-800 nm with the inline PDA detector. Mass spectrometry was conducted on a quadrupole ion tunnel mass spectrometer equipped with a Z-spray ESI source. Explant protein levels were determined by the Lowry method, using bovine γ-globulin as the standard protein [Lippman et al., 1993].

### Example 1

It was desired to evaluate the effect of pH and concentration of FBR on the efficacy of the compositions and methods of the present invention. Accordingly, various mucoadhesive berry gels varying in final pH and % FBR were prepared for various analyses and in-vivo studies. Five studies were completed as follows: 1) pH 6.5 gels with 5% w/w FBR for a human pharmacokinetic study, 2) pH 6.5 gels with 5% and 10% w/w FBR for anthocyanin uptake studies in human mucosa explant tissue, 3) pH 4.5, 5.5, 6.5, and 7.5 gels with 10% w/w FBR to determine anthocyanin stability at 1 week at 4° C., 25° C., and 40° C., 4) pH 3.5 and pH 4.0 gels with 10% w/w FBR to determine anthocyanin stability over 1 month at 4° C. and 25° C., and 5) pH 3.5 and pH 6.5 gels with 10% w/w FBR to compare difference in anthocyanin uptake into human mucosa explant tissue.

All gels were prepared in stainless steel vessels using a Caframo Stirrer Model BDC-1850 (Wiarton, Ontario, Canada) with attached metal blade. To the required amount of purified water stirring in the vessel, Noveon AA1 and Carbopol 971P were added slowly and allowed to fully hydrate for at least 1 hr. Next, glycerin, 2-phenoxyethanol, and benzyl alcohol were added followed by EDTA. The gel was allowed to mix for another 1 hr. Sodium hydroxide (2.5 N) was added to raise the pH of the gel to pH 7.5. Placebo gels were semi-transparent and homogenous in appearance. Finally, powdered FBR was added in different amounts to produce final concentrations of FBR of either 5% or 10% w/w. Either 2.5 N NaOH or 2.33 N HCl was added to adjust to the desired pH. Purified water was then added to q.s. the gels to weight.

Gels having a final pH in the range of pH 4.5 to 7.5 had the following final concentration of excipients: Noveon AA1 (0.675% w/w), Carbopol 971P (0.788% w/w), glycerin (0.9% w/w), 2-phenoxyethanol (0.9% w/w), benzyl alcohol (0.9% w/w), and EDTA (0.09% w/w). Two adjustments were subsequently made for gels having a fmal pH of either pH 3.5 or 4.0. First, the amounts of Noveon AA1 and Carbopol 971P needed to produce a final berry gel having comparable viscosity at lower pH were significantly increased. Second, the concentration of all other excipients was increased by 10% to adjust for the dilutive effects of adding powdered FBR at the level of 10% w/w. Thus, gels having a fmal pH of either pH 3.5 or 4.0 had the following final concentration of excipients: Noveon AA1 (1.35% w/w), Carbopol 971P (1.575% w/w), glycerin (1.0% w/w), 2-phenoxyethanol (1.0% w/w), benzyl alcohol (1.0% w/w), and EDTA (0.1% w/w).

All prepared gels were stored at 2-8° C. (unless stated otherwise), sealed and protected from light in 10 g white Aluminum Tubes, sealed neck with Morton PE 1090 liner (Montebello Packaging; Ontario, Canada). Gels were characterized for pH, viscosity, osmolality, and anthocyanin content. The pH of gels was measured using an Orion pH meter Model 520A. Gel viscosity was determined using a Brookfield Cone & Plate Rheometer Model RVDV III+ (Brookfield Engineering, Middleboro, Mass.) at 25° C. for 1 min at an RPM of 4 using Spindle CPE-52. The osmolality was measured using a Fiske 110 Freezing-Point Depression Osmometer. The Microbial Limits Test <61> in USP 29 was used to determine the total microbial count of the gels, and for the presence of yeasts and molds.

With reference to a representative embodiment of the FBR gel of the present invention as shown in Table 1, gels were prepared by adding FBR to semi-transparent and homogenous placebo gels to achieve a final FBR concentration of up to 10% w/w. For unbuffered placebo gels, the addition of FBR resulted in a lowering of the gel pH in proportion to the amount of FBR added. For example, the addition of 10% FBR to a placebo gel with a pH of 6.9 resulted in a final pH of the 10% w/w gel of 5.9. However, the pH of the 10% w/w berry gel was easily adjusted to the desired pH. The drop of pH after the addition of FBR was due to the naturally low pH of the FBR, since a 10% w/w slurry of the FBR in water had a pH of 4.3.

TABLE 1
Composition of a Representative Mucoadhesive
Berry Gel (pH 3.5) for Mucosal Application

| Ingredient (grade) | Function | % w/w |
|---|---|---|
| Purified Water | Base vehicle | qs to 100% |
| Noveon AA1 (NF) | Mucoadhesive | 1.35 |
| Carbopol 971P (NF) | Mucoadhesive polymer/ | 1.575 |
| | thickener | |
| Glycerin (USP) | Emollient | 1.0 |
| Edetate Disodium | Metal chelator (anti-oxidant) | 0.1 |
| (EDTA) (USP) | | |
| 2-Phenoxyethanol (BP) | Anti-microbial preservative | 1.0 |
| Benzyl alcohol (USP) | Anti-fungal preservative | 1.0 |
| 2.5 N NaOH or 2.33 N HCl | Adjust to final pH of 3.5 | as needed |
| Freeze-dried black | Active (5% or 10% w/w) | 5.0 or 10 |
| raspberries (FBR) | | |

The addition of FBR to placebo gels resulted in a red to black homogenous gel with a viscosity target range from 10,000 to 20,000 cP. The addition of FBR to placebo gel did not significantly affect final gel viscosity.

### Example 2

To quantify and evaluate stability of the anthocyanin composition of FBR gels prepared as described in Example 1, a reversed-phase HPLC assay was developed and partially validated. The HPLC assay was developed based on a previously described assay by Tian et al. [2005]. For the assay, a Thermoquest HPLC system with a UV6000LP photodiode array detector, a Water's Symmetry C18 column (3.9×150 mm, 5 μm), and a Phenomenex Security Guard with C18 cartridge were employed. FBR standards (0-2 mg/mL) were prepared by dissolving FBR in aqueous 1% formic acid (mobile Phase A) and vortexing to completely dissolve the powder. The slightly cloudy standards were filtered through a 0.45 μm hydrophilic PTFE syringe filter into a HPLC vial. A linear gradient (flow rate of 1.0 mL/min) of mobile Phase A (1% formic acid) and mobile phase B (100% acetonitrile) was utilized at a column temperature of 40° C. as follows: 93% A (isocratic) for 0-2 min, 93% A to 89% A (linear gradient) for 2-18 min, 89% A to 0% A (linear gradient) for 18-19 min, 0% A (isocratic) for 19-25 min, 0% A to 93% A (linear gradient) for 25-26 min, and 93% A (isocratic) for 26-35 min. The total run time was 35 min. The injection volume was 10 μL and the detection wavelength for the anthocyanins was 520 nm. The FBR gels were analyzed by adding 100 mg of gel to a 10 mL volumetric flask with aqueous 1% formic acid followed by the subsequent steps as described above.

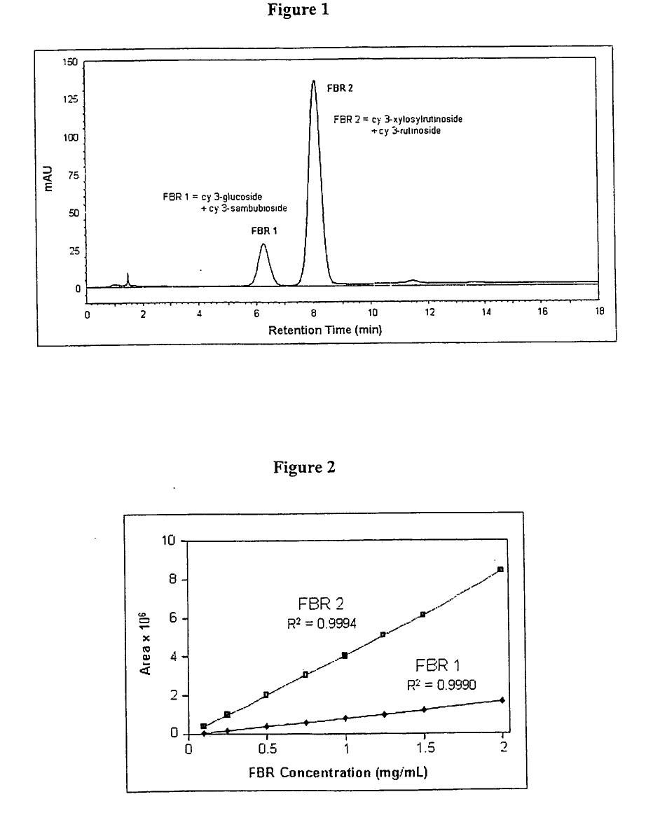

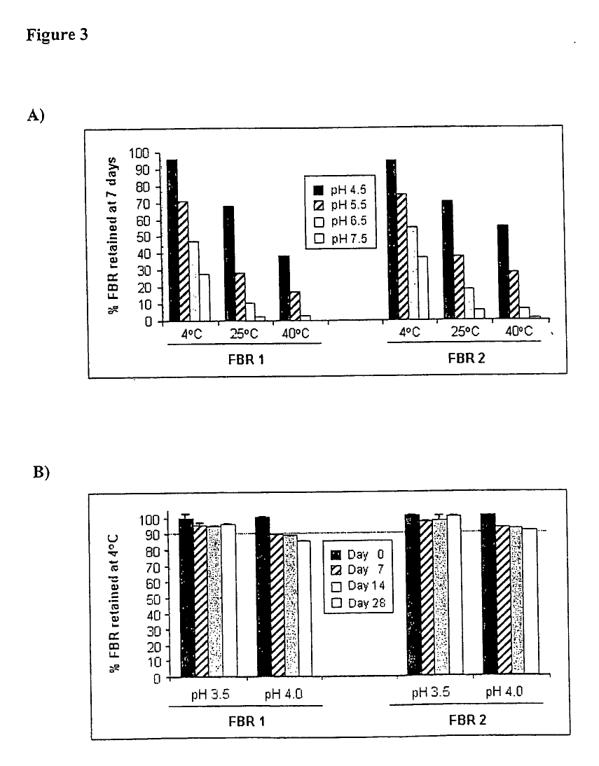

With reference to the representative standard chromatogram presented in FIG. 1, two peaks termed FBR 1 and FBR 2 were seen. These two peaks appear to correspond to the four peaks shown in Tian et al. [2005]. It was subsequently determined using LC-MS (see below) that FBR 1 correspond to cyanidin 3-glucoside and cyanidin 3-sambubioside and FBR 2 correspond to cyanidin 3-xylosylrutinoside and cyanidin 3-rutinoside. For initial development of the HPLC assay, a 1% formic acid mobile phase was used instead of 10% formic acid as previously described by Tian et al. to conserve the stability and integrity of the C18 column. As shown in FIG. 2, the standard curve for both FBR 1 and FBR 2 showed excellent linearity over the range of 50 μg/mL to 2000 μg/mL. System suitability for the assay calculated by averaging the peak areas of all standard injections showed that the relative standard deviation (RSD) of the assay was less than 1%. In addition, the recovery of anthocyanins (both FBR 1 and FBR 2) from the gels was greater than 99%.

Initial studies indicated that lower pH and lower storage temperatures were more favorable for FBR1 and FBR2 peak stability. FIG. 3A shows the percent detected for 10% berry gels at various pH values after 24 hr storage at 4, 25, and 40° C. For both FBR 1 and FBR2, there was a very strong correlation between pH storage temperatures in terms of peak retention. It is well known that anthocyanins exist at low pH as a flavylium cation, which is their naturally occurring form. The flavylium cation is highly electron deficient and intensely colored (red or orange) at a pH below about pH 4.5. At higher pHs, anthocyanins exist as either a quinoidal base, carbinol pseudobase, or the chalcone pseudobase. Thus, in addition to being less stable at higher pH, the flavylium cation is known to be less stable at increased temperatures [Rubinskiene et al., 2005; Morais et al., 2002].

Subsequent stability studies were performed using four gels formulated at 3.5 and 4.0, with and without 0.1% w/w ascorbic acid as an additional anti-oxidant. The results (see FIG. 3B) demonstrate that the pH 3.5 gel provided excellent stability for FBR1 (96.0±0.8%) and FBR 2 (100.2±0.4%) over a 1 month storage period at 4° C. Gels at pH 4, gels stored at 25° C., and gels with 0.1% ascorbic acid (data not shown) demonstrated less stability for both FBR1 and FBR2. The results of the second stability study demonstrated that pH 3.5 provided for the greatest storage stability of FBR 1 and FBR 2 when 10% FBR was formulated in the gel and stored at 4° C.

### Example 3

Next, evaluation of anthocyanin uptake into human oral mucosa from FBR gels prepared as described in Example 1 was undertaken. For in-vivo studies, participation of human subjects was conducted in accordance with an IRB approved protocol. None of the human subjects had a diet rich in anthocyanin compounds prior to participation in either the pharmacokinetic or tissue explant studies. For the phamacokinetic analyses, nine consenting adult volunteers dried the anterior floor of their mouth, placed 1.0 g of 5% berry gel (pH 6.5), and massaged the gel in place for 30 seconds to facilitate uptake. Two minutes after gel application saliva was collected for the next three minutes. Peripheral blood was drawn five minutes after gel application. Following clotting, sera samples were collected, and both the sera and saliva samples were stored at −80° C. until LC-MS analysis as described below.

The human pharmacokinetic studies which used the 5% berry gel (pH 6.5) demonstrated that bioadhesive berry gels were readily absorbed into human oral mucosa (see Table 2). All nine participants had detectable anthocyanin levels of three anthocyanins (cyanidin 3-rutinoside, cyanidin 3-xylosylrutinoside, cyanidin 3-glucoside) in either their saliva or blood, and 4 donors had detectable levels of two anthocyanins (cyanidin 3-rutinoside and cyanidin 3-xylosylrutinoside) in their peripheral blood. Saliva from eight of the nine donors contained detectable levels of either two (cyanidin 3-rutinoside and cyanidin 3-xylosylrutinoside-4 donors) or three anthocyanins (4 donors).

### Example 4

Tissue explants, specifically human oral mucosal explants, were used to determine the in vitro effects of FBR content and pH on anthocyanin uptake. Human oral tissues for the tissue explant studies were obtained from consenting volunteers who were undergoing elective oral surgery procedures. To confirm “no pathologic diagnosis”, a portion of each of these tissues was submitted for light microscopic evaluation. All tissue donors were systemically healthy and did not use tobacco products. Tissues for explant experiments were immediately placed in an artificial saliva transport medium, which also served as the incubation medium for the gel absorption studies. The composition of the artificial saliva used for tissue transport and incubation was (final concentrations): sodium carboxymethylcellulose (0.5%), sorbitol (165 mM), NaCl (14 mM), KCl (16 mM), calcium chloride dehydrate (1.0 mM), MgCl₂ (0.63 mM), Trizma hydrochloride (2.0 mM).

Tissues from eight donors were used for preliminary studies to compare the penetration of anthocyanins from the 5% berry gel and the 10% FBR gel, both at a final pH of 6.5. In order to account for heterogeneity among human donors, tissue samples were hemisected. One tissue portion treated with 5% and the other with 10% FBR gels. One gram of gel (containing either 5% or 10% w/w FBR) was placed on the epithelial surface of the explant, followed by gently rubbing of the site for 30 seconds to facilitate uptake. Explants were then placed into artificial saliva, incubated for 30 minutes (37° C., 5% CO₂), and then frozen at −80° C. until HPLC-mass spectroscopy analyses as previously reported [Rodrigo et al., in press]. Tissue preparation for HPLC analyses consisted of homogenization of the entire oral mucosal specimen, removal of an aliquot for sample protein level determination, followed by the addition of collagenase type II (0.1 mg/mL, GIBCO, Grand Island, N.Y.) and 1% formic acid. Samples were then placed on a Sep Pack (Millipore, Billerica, Mass.), eluted with methanol, and dried under argon gas.

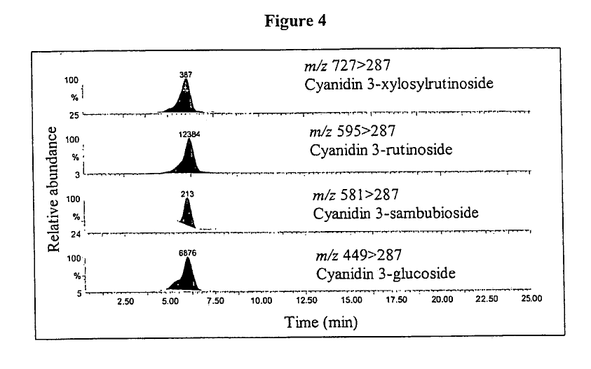

Comparing the ability of 5% relative to 10% berry gels (both adjusted to a pH of 6.5) to absorb into human oral mucosa explants, it was found that the 10% berry gel delivered detectable levels of all four anthocyanin compounds (see FIG. 4) into all eight of the tissue explants evaluated. In contrast, only two anthocyanins (cyanidin 3-rutinoside and cyanidin 3-xylosylrutinoside) were identified in tissues treated with the 5% gel formulations.

TABLE 2
Anthocyanin Concentration (pmol) in Saliva and Peripheral Blood Five Minutes
After Topical Application of a 5% FBR Gel, pH 6.5 to the Floor of the Mouth

| Patient | | SML 101 | SML 102 | SML 103 | SML 104 | SML 105 | SML 106 | SML 107 | SML 108 | SML 109 |
|---|---|---|---|---|---|---|---|---|---|---|
| Gender | | F | M | M | F | M | M | M | F | M |
| Age | | 50 | 62 | 29 | 48 | 42 | 59 | 33 | 56 | 48 |
| Current Smoking Status | | Non- | Non- | Non- | Non- | Non- | Non- | Non- | Non- | Non- |
| | | Smoker | Smoker | Smoker | Smoker | Smoker | Smoker | Smoker | Smoker | Smoker |
| Saliva | cyanidin 3- | 3.5 | 1.7 | ND | 4.6 | 8.3 | 6.1 | 9.3 | 4.2 | 2.2 |
| (pmol/ml) | rutinoside | | | | | | | | | |
| | cyanidin 3- | 2.0 | 0.5 | ND | 1.5 | 2.3 | 1.9 | 1.7 | 1.0 | 1.2 |
| | xylosylrutinoside | | | | | | | | | |
| | cyanidin 3- | ND | ND | ND | 11.2 | 9.7 | ND | 19.7 | ND | 22.8 |
| | glucoside | | | | | | | | | |
| Blood | cyanidin 3- | ND | ND | 2.3 | ND | 7.4 | 5.6 | 5.8 | ND | ND |
| (pmol/ml) | rutinoside | | | | | | | | | |
| | cyanidin 3- | ND | ND | 0.6 | ND | 1.1 | 0.8 | 1.3 | ND | ND |
| | xylosylrutinoside | | | | | | | | | |
| | cyanidin 3- | ND | ND | ND | ND | ND | ND | ND | ND | ND |
| | glucoside | | | | | | | | | |

Additional oral mucosal explant studies were conducted to compare the effect of FBR gel pH on absorption into oral mucosal explants. Tissues from eleven donors were used for these studies, and handling was as described above with the exception that all gels contained 10% w/w FBR and were formulated at a final pH of 3.5 or 6.5. In contrast to the human pharmacokinetic evaluations, tissue explant studies showed the presence of all four anthocyanins contained in FBR, inclusive of cyanidin 3-sambubiocide. Further, these data demonstrated approximately 2-3 fold higher levels of the respective anthocyanin in tissue explants relative to levels determined in either saliva or blood. As shown in Table 3, studies to compare the effect of gel pH on tissue anthocyanin uptake suggested greater penetration with the pH 6.5 gels. Four donors' tissues showed increased penetration of all four anthocyanin compounds with the pH 6.5 relative to the pH 3.5 gels. Notably, there were large variations that reflected both the anthocyanin under evaluation as well as the penetrability of the individual donors' tissues. Further, depending upon the anthocyanin compound under analysis, there were between 4 to 6 tissue donors who consistently showed greater penetration with the pH 3.5 gel. Large differences (up to 15-fold) in detectable anthocyanin levels were found in the gel treated donor tissues; findings that likely reflect the extensive heterogeneity within human tissues that affects tissue penetrative abilities. Finally, although these data demonstrate trends towards enhanced tissue penetration with the higher pH 6.5 berry gel, due to large inter- and intra-donor standard deviations, these differences were not statistically significant.

### Example 5

A gel substantially as described in Example 1 is prepared, with the exception that a solvent-extracted FBR preparation is used. Solvent extraction is conducted substantially as described in copending U.S. utility patent application Ser. No. 10/951,413. Briefly, 400 grams of FBR are extracted in 4,000 ml of methanol or ethanol overnight at room temperature. The solvent-extracted preparations are then concentrated under vacuum, such that the concentrated, solvent-extracted FRB preparation typically represents approximately 55% of the starting freeze-dried material. The concentrated solvent-extracted FBR preparations is then incorporated into a mucoadhesive gel substantially as described in Example 1.

### Example 6

In another embodiment of the present invention, a solvent-extracted FBR preparation is used to formulate a mucoadhesive gel preparation. Solvent extraction is conducted substantially as described in Hecht et al. [in press]. Briefly, FBR (25 g) prepared as described in Example 1 is sequentially extracted with pentane, methylene chloride, absolute ethanol, and water. The FBR preparation is placed in a Soxhlet extraction thimble and extracted with 400 ml pentane for 16 hr. Fresh pentane (400 ml) is added, and the procedure repeated for 16 hr. The process is repeated with the same volumes of methylene chloride and absolute ethanol. Next, the residue is removed from the thimble and sonicated 3 times with 100 ml aliquots of water (30 min. sonications). Extracts from each solvent extraction are combined, and the solvents removed by rotary evaporation. In the case of the water-sonicated residue, water is removed by lyophilization. The solvent-extracted FRB preparation typically represents approximately 18% of the starting freeze-dried material. The concentrated solvent-extracted FBR preparation is then incorporated into a mucoadhesive gel substantially as described in Example 1.

### Example 7

A different solvent extraction procedure is used to formulate a mucoadhesive FBR gel preparation. Solvent extraction is conducted substantially as described in Hecht et al. [in press]. Briefly, FBR (25 g) prepared as described in Example 1 is placed in a 1 L Erlenmeyer flask and sequentially extracted with stirring at room temperature with 3 400 ml portions each of pentane, methylene chloride, absolute ethanol, and water. An explosion-proof mechanical stirrer (Caframo, Ltd., Wiarton, Ontario, Canada) is used. Each extraction is carried out for 16 hr. The solvent-extracted FRB preparation typically represents approximately 18% of the starting freeze-dried material. The concentrated solvent-extracted FBR preparation is then incorporated into a mucoadhesive gel substantially as described in Example 1.

TABLE 3
Effect of Final Gel pH on the Anthocyanin Uptake into Human Oral Mucosa Explant Tissues

| Tissue | Cyanidin 3-glucoside | | | Cyanidin 3-sambubioside | | | Cyanidin 3-rutinoside | | | Cyanidin 3-xylosylrutinoside | | |
|---|---|---|---|---|---|---|---|---|---|---|---|---|
| Explant | (pmol per mg tissue) | | | (pmol per mg tissue) | | | (pmol per mg tissue) | | | (pmol per mg tissue) | | |
| Sample | Gel | Gel | % change | Gel | Gel | % change | Gel | Gel | % change | Gel | Gel | % change |
| No. | pH 3.5 | pH 6.5 | 3.5 to 6.5 | pH 3.5 | pH 6.5 | 3.5 to 6.5 | pH 3.5 | pH 6.5 | 3.5 to 6.5 | pH 3.5 | pH 6.5 | 3.5 to 6.5 |
| 001 | 128.1 | 296.6 | 131.6 | 7.4 | 30.9 | 319.4 | 116.4 | 668.3 | 474.1 | 14.2 | 88.1 | 522.5 |
| 002 | 13.6 | 18.5 | 36.2 | 0.6 | 2.9 | 427.3 | 9.3 | 67.8 | 627.2 | 1.2 | 10.3 | 747.1 |
| 003 | 14.4 | 12.0 | −16.6 | 1.8 | 1.4 | −19.7 | 34.3 | 29.2 | −14.8 | 5.7 | 6.4 | 12.1 |
| 004 | 1.6 | 12.0 | 653.5 | 1.3 | 0.4 | −66.4 | 26.3 | 9.4 | −64.4 | 3.6 | 1.3 | −65.1 |
| 005 | 32.0 | 52.9 | 65.3 | 0.3 | 0.1 | −61.8 | 3.5 | 2.8 | −20.0 | 0.9 | 0.5 | −45.9 |
| 006 | 31.5 | 60.3 | 91.3 | 2.3 | 10.1 | 330.8 | 45.7 | 211.1 | 362.1 | 9.9 | 35.5 | 258.5 |
| 007 | 17.0 | 22.8 | 34.5 | 0.4 | 0.4 | −14.6 | 5.6 | 8.1 | 45.1 | 1.1 | 1.0 | −9.4 |
| 008 | 33.2 | 19.7 | −40.7 | 0.0 | 1.9 | — | 0.4 | 35.1 | 8905.1 | 0.0 | 6.3 | — |
| 009 | 21.2 | 45.3 | 113.9 | 2.9 | 10.3 | 256.9 | 43.9 | 207.4 | 395.1 | 9.9 | 41.3 | 318.4 |
| 010 | 13.9 | 4.6 | −66.9 | 2.0 | 0.1 | −92.9 | 37.1 | 3.2 | −91.4 | 6.7 | 0.5 | −91.9 |
| 011 | 18.9 | 7.5 | −60.1 | 2.1 | 0.0 | −100 | 28.7 | 0.4 | −98.7 | 6.0 | 0.00 | −100 |
| Avg. ± | 29.6 ± | 50.2 ± | 85.6 ± | 1.9 ± | 5.3 ± | 89.0 ± | 31.9 ± | 113.0 ± | 956.3 ± | 5.4 ± | 17.4 ± | 140.6 ± |
| SEM | 10.3 | 25.3 | 60.4 | 0.6 | 2.8 | 60.3 | 9.8 | 60.3 | 797.9 | 1.4 | 8.3 | 85.6 |

### Example 8

A mucoadhesive FBR gel preparation is prepared using ethanol extraction substantially as described in Hecht et al. [in press] and in Examples 6-7. Briefly, FBR (25 g) prepared as described in Example 1 is placed in a 1 L Erlenmeyer flask and extracted with stirring at room temperature with 3 400 ml portions of absolute ethanol. Each extraction is carried out for 16 hr. The solvent-extracted FRB preparation typically represents approximately 50% of the starting freeze-dried material. The concentrated solvent-extracted FBR preparations are then incorporated into a mucoadhesive gel substantially as described in Example 1.

### Example 9

A 5 kg batch of the 10% FBR Gel, pH 3.5 was prepared using the formula described in Table 1. The gel was placed in Aluminum Tubes, sealed neck with Morton PE 1090 liner (Montebello Packaging; Ontario, Canada) tubes and stored under controlled conditions at 4° C. (2-8° C.). As determined by the Microbial Limits Test <61> in USP 29, the total microbial count of the gel was 255 colony forming units (cfu)/g and was found to be free of Escherichia coli, Salmonella, Staphylococcus aureus, Pseudomonas aeruginosa. The viscosity was measured using a Brookfield RVDV III Cone & Plate Rheometer using spindle 52 at 4 rpm at 25° C. for 1 min. The osmolality was measured using a Fiske 110 Freezing-Point Depression Osmometer. The pH was measured using a Orion pH meter Model 520A.

TABLE 4
Stability of 10% FBR Gel, pH 3.5 stored for 6 months at 4° C.

| | | | | | Osmolality |
|---|---|---|---|---|---|
| Time Point | % FBR1 | % FBR2 | | Viscosity | (mOsm/kg |
| (Months) | (% RSD) | (% RSD) | pH | (cP) | H₂O) |
| 0 | 94.4 (2.4) | 97.8 (3.0) | 3.4 | 17,405 | not tested |
| 1 | 86.0 (2.5) | 91.2 (3.0) | 3.4 | 17,595 | not tested |
| 2 | 84.8 (2.0) | 92.5 (2.5) | 3.4 | 19,489 | not tested |
| 3 | 78.1 (0.2) | 86.8 (1.7) | 3.4 | 12,642 | not tested |
| 6 | 76.1 (3.9) | 86.9 (2.6) | 3.4 | 17,099 | 811 ± 9 |

After 6 months storage at 4° C., the gel was found to contain no mold. Yeast present in the gel was identified as Candida colliculosa and was present at 300 cfu/g as determined by the Microbial Limits Test <61> in USP 29.

### Example 10

A 10% FBR gel was prepared substantially as described in Example 9, with the pH adjusted to 3.5. Fourteen patients with either stage I, II, or III dysplasia and ten control patients were treated with 2 grams of the gel per lesion per day for a period of 42 days. The control participants applied the same amount to gel to a specified site (lateral tongue) that consisted of clinically normal tissue. Patients were monitored for clinical assessment and light microscopic diagnosis to determine lesion size, diminished leukoplakia (white appearing tissue) or erythroplakia (velvet red appearing tissue). No participant experienced any observable adverse effects, such as mucositis and/or delayed wound healing. Microscopic evaluation revealed that 6 of the 14 dysplasia patients exhibited a decrease in disease severity, assessed by histologic grade.

The present disclosure therefore shows that an effective composition is provided for the chemoprevention of certain types of cancer, especially oral cancers. Furthermore, the concentrating processes described herein concentrate the berries bioactive constituents at least ten-fold, introducing a greater chemopreventive impact within a particular gel formulation. The local delivery formulations described for the compositions of the present invention provide a significant pharmacologic benefit; specifically the ability to obtain therapeutically relevant local levels as well as rapid absorption kinetics for systemic benefits, without development of systemic complications. Anthocyanin detection in the saliva following topical gel application supports the premise that bioadhesive gels can provide field coverage throughout the mouth. The surprising detection of anthocyanins in peripheral blood within 5 minutes of FBR gel application provides even more compelling evidence that the gel formulation efficiently delivers bioactive anthocyanin compounds to the target tissue site. In addition, the explant studies suggested that gel formulations provide a pharmacologic advantage to the target tissue site (higher anthocyanin levels in explants relative to either saliva or blood). Although it is difficult to compare these results to those of several human studies investigating the oral absorption of much larger quantities of anthocyanins in food or beverages [Kay et al., 2005; Bitsch et al., 2004; Matsumoto et al., 2001] it is fair to conclude that the transmucosal absorption of anthocyanins from the berry gel was relatively rapid and potentially greater from the mouth compared to the oral absorption achieved by dietary supplementation.

The foregoing description of the preferred embodiment of this invention has been presented for purposes of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

### Citations of Literature

Bitsch I, Janssen M, Netzel M, Strass G, Frank T. Bioavailability of anthocyanidin-3-glycosides following consumption of elderberry extract and blackcurrant juice. Int J Clin Pharmacol Ther. 2004, 42(5):293-300.
Braakhuis B J, Tabor M P, Leemans C R, van der Waal I, Snow G B, Brakenhoff R H. Second primary tumors and field cancerization in oral and oropharyngeal cancer: molecular techniques provide new insights and definitions. Head Neck 24: 198-206 (2002).
Casto B C, Kresty L A, Kraly C L, Pearl D K, Knobloch T J, Schut H A, Stoner G D, Mallery S R, Weghorst C M. Chemoprevention of oral cancer by black raspberries. Anticancer Res 22: 4005-4016 (2002).
Hecht S S, Huang C, Stoner G D, Li J, Kenney P M J, Sturla S J, Carmella S G. Identification of cyanidin glycosides as constituents of freeze-dried black raspberries which inhibit anti-benzo[a]pyrene-7,8-diol-9,10-epoxide induced NFκB and AP-1 activity. In Press: Carcinogenesis.
Hu C, Zawistowski J, Ling W, Kitts D D. Black Rice (Oryza sativa L. Indica) Pigmented Fraction Suppresses both Reactive Oxygen Species and Nitric Oxide in Chemical and Biological Model Systems. J Agric Food Chem 51: 5271-5277 (2003).
Huang C, Huang Y, Li J, Hu W, Aziz R, Tang M-S, Sun N, Cassady J, Stoner G D. Inhibition of Benzo(a)pyrene Diol-Epoxide-induced Transactivation of Activated Protein 1 and Nuclear Factor κB by Black Raspberry Extracts. Cancer Res 62: 6857-6863 (2002).
Jemal A. Murray T, Ward E, Samuels A, Tiwari R C, Ghafoor A, Feuer E J, Thun M J. Cancer Statistics, 2005. CA A Cancer Journal for Clinicians. 55: 10-11 (2005).
Katsube N, Iwashita K, Tsushida T, Yamaki K, Kobori M. Induction of apoptosis in cancer cells by Bilberry (Vaccinium myrtillus) and the anthocyanins. J Agric Food Chem 51: 68-75 (2003).
Kay C D, Mazza G J, Holub B J. Anthocyanins exist in the circulation primarily as metabolites in adult men. J Nutr. 2005. 135(11):2582-8.
Kresty A, Morse M A, Morgan C, Carlton P S, Lu J, Gupta A, Blackwood M, Stoner G D. Chemoprevention of esophageal tumorigenesis by dietary administration of lyophilized black raspberries. Cancer Res 61: 6112-6119 (2001).
Lippman S M, Batsakis J G, Toth B B et al. Comparison of low-dose isoretinoin with beta-carotene to prevent oral carcinogenesis. N Eng J Med 328: 15-20, 1993.
Liu M, Li X Q, Weber C, Lee C Y, Brown J, Liu R H. Antioxidant and antiproliferative activities of raspberries. J Agric Food Chem 50: 2926-2930 (2002).
Malone J P, Stephens J A, Grecula J C, Rhoades C A, Ghaheri B A, Schuller D E. Disease control, survival, and functional outcome after multimodal treatment for advanced-state tongue base cancer. Head Neck 26: 561-572 (2004).
Matsumoto H, Inaba H, Kishi M, Tominaga S, Hirayama M, Tsuda T. Orally administered delphinidin 3-rutinoside and cyanidin 3-rutinoside are directly absorbed in rats and humans and appear in the blood as the intact forms. J Agric Food Chem. 2001 49(3):1546-51.
Morais H, Ramos C, Forgacs E, Cserhati T, Oliviera J. Influence of storage conditions on the stability of monomeric anthocyanins studied by reversed-phase high-performance liquid chromatography. J Chromatogr B Analyt Technol Biomed Life Sci. 2002, 25;770(1-2):297-301.
Papadimitrakopoulou V A, Clayman G L, Shin D M, Myers J N, Gillenwater A M, Goepfert H, El-Naggar A K, Lewin J S, Lippman S M, and Hong W K. Biochemoprevention for dysplastic lesions of the upper aerodigestive tract. Arch Otolaryngol Head Neck Surg 125: 1083-1089 (1999).
Pathak K A, Gupta S, Talole S, Khanna V, Chaturvedi P, Deshpande M S, Pai P S, Chaukar D A, D'Cruz A K. Advanced squamous cell carcinoma of lower gingivobuccal complex: patterns of spread and failure. Head Neck 27: 597-602 (2005).
Rodrigo K A, Rawal Y, Renner R J, Schwartz S J, Tian Q, Larsen P E, Mallery S R. Suppression of the tumorigenic phenotype in squamous cell carcinoma cells by an ethanol extract derived from freeze-dried black raspberries. In Press: Nutrition and Cancer.
Rubinskiene M, Jasutiene I, Venskutonis P R, Viskelis P. HPLC determination of the composition and stability of blackcurrant anthocyanins. J Chromatogr Sci. 2005, 43(9):478-82.
Schwartz G J, Mehta R H, Wenig B L, Shaligram C, Portugal L G. Salvage treatment for recurrent squamous cell carcinoma of the oral cavity. Head Neck 22: 34-41 (2000).
Stoner Gary D, Sardo C, Apseloff G, Mullet D, Wargo W, Pound V, Singh A, Sanders J, Aziz R, Casto B, Sun X L. Pharmacokinetic of Anthocyanins and Ellagic Acid in Healthy Volunteers Fed Freeze-Dried Black Raspberries for 7 Days. J Clin Pharmacol. 45: 1153-1164 (2005).
Tian Q, Giusti M M, Stoner G D, and Schwartz S J. Characterization of a new anthocyanin in black raspberries (Rubus occidentalis) by liquid chromatography electrospray ionization tandem mass spectrometry. Food Chemistry 94: 465-468 (2005).
Toma A, Bonelli L, Sartoris A, Mira E, Antonelli A, Beatrice F, Giordano C, Benazzo M, Caroggio A, Cavalot A L, Gandolfo S, Garozzo A, Margarino G, Schwnone G, Spadini N, Sirotova Z, Zibordi F, Balazarini F, Serafini I, Miani P, Cortesina G. 13-cis retinoic acid in head and neck cancer chemoprevention: results of a randomized trial from the Italian Head and Neck Chemoprevention Study Group. Oncol Rep 11: 1297-1305 (2004).

What is claimed is:

1. A composition for chemoprevention of an oral cancer or precancerous condition, comprising an isolated berry preparation admixed with a bioadhesive carrier.

2. The composition of claim 1, wherein the isolated berry preparation comprises at least one anthocyanin in an amount effective for chemoprevention.

3. The composition of claim 2, wherein the isolated berry preparation is derived from the group consisting of strawberry, raspberry, red raspberry, black raspberry, and a combination thereof.

4. The composition of claim 3, wherein the isolated berry preparation is prepared by physically disrupting and concentrating a quantity of berries.

5. The composition of claim 4, wherein the physically disrupted berries are concentrated by at least one of freeze-drying and solvent extraction.

6. The composition of claim 1, wherein the isolated berry preparation admixed with a bioadhesive carrier is adjusted to a pH of from about 3.0 to about 4.5 for storage.

7. The composition of claim 6, wherein the isolated berry preparation admixed with a bioadhesive carrier is adjusted to a pH of about 3.5 for storage.

8. The composition of claim 5, including from about 1% (w/w) to about 10% (w/w) concentrated berry preparation.

9. The composition of claim 8, including from about 5% (w/w) to about 10% (w/w) concentrated berry preparation.

10. The composition of claim 1, wherein the bioadhesive carrier is a mucoadhesive gel adapted for transmucosal delivery of the isolated berry preparation.

11. The composition of claim 10, wherein the mucoadhesive gel is provided at a final viscosity of from about 10,000 cP to about 20,000 cP.

12. A method for chemoprevention of an oral cancer or precancerous condition, comprising topically administering to an individual in need of such chemoprevention an isolated berry preparation admixed with a bioadhesive carrier.

13. The method of claim 12, wherein the isolated berry preparation admixed with a bioadhesive carrier is administered to an interior of an oral cavity of the individual.

14. The method of claim 12, wherein the isolated berry preparation comprises at least one anthocyanin in an amount effective for chemoprevention.

15. The method of claim 14, wherein the isolated berry preparation is derived from the group consisting of strawberry, raspberry, red raspberry, black raspberry, and a combination thereof.

16. The method of claim 15, wherein the isolated berry preparation is prepared by the steps of: (a) physically disrupting a quantity of berries; and (b) concentrating the physically disrupted berries.

17. The method of claim 16, wherein the physically disrupted berries are concentrated by at least one of freeze-drying and solvent extraction.

18. The method of claim 14, wherein the isolated berry preparation admixed with a bioadhesive carrier is adjusted to a pH of from about 3.0 to about 4.5 for storage.

19. The method of claim 18, wherein the isolated berry preparation admixed with a bioadhesive carrier is adjusted to a pH of about 3.5 for storage.

20. The method of claim 17, wherein the isolated berry preparation admixed with a bioadhesive carrier is formulated to include from about 1% (w/w) to about 10% (w/w) concentrated berry preparation.

21. The method of claim 20, wherein the isolated berry preparation admixed with a bioadhesive carrier is formulated to include from about 5% (w/w) to about 10% (w/w) concentrated berry preparation.

22. The method of claim 14, including the step of admixing the isolated berry preparation with a bioadhesive carrier that is a mucoadhesive gel adapted for transmucosal delivery of the isolated berry preparation.

23. The method of claim 22, including the step of providing the mucoadhesive gel at a viscosity of from about 10,000 cP to about 20,000 cP.

24. A method for stabilizing an anthocyanin contained in an isolated berry preparation, comprising:
admixing the isolated berry preparation with a bioadhesive carrier to form a mixture; and
adjusting the mixture to a pH of from about 3.0 to about 4.5.

25. The method of claim 24, wherein the pH of the mixture is adjusted to about 3.5.

26. The method of claim 24, wherein the isolated berry preparation is derived from the group consisting of strawberry, raspberry, red raspberry, black raspberry, and a combination thereof.

27. The method of claim 26, wherein the isolated berry preparation is prepared by the steps of: (a) physically disrupting a quantity of berries; and (b) concentrating the physically disrupted berries.

28. The method of claim 27, wherein the physically disrupted berries are concentrated by at least one of freeze-drying and solvent extraction.

29. The method of claim 24, including the step of admixing the isolated berry preparation with a bioadhesive carrier that is a mucoadhesive gel.

30. A method for preparing a composition for chemoprevention of an oral cancer or precancerous condition, comprising:
physically disrupting a quantity of berries;
concentrating the physically disrupted berries; and
admixing the berry concentrate with a bioadhesive carrier.

31. The method of claim 30, wherein the physically disrupted berries are concentrated by at least one of freeze-drying or solvent extraction.

32. The method of claim 31, further including the step of adjusting a pH of the bioadhesive carrier containing the berry concentrate to from about 3.0 to about 4.5 for storage.

33. The method of claim 32, including the further step of adjusting the pH of the bioadhesive carrier containing the berry concentrate to about 3.5 for storage.

34. The method of claim 30, further including the step of deriving the isolated berry preparation from the group consisting of strawberry, raspberry, red raspberry, black raspberry, and combinations thereof.

35. The method of claim 31, including the step of admixing from about 1% (w/w) to about 10% (w/w) concentrated berry preparation with the bioadhesive carrier.

36. The method of claim 35, including the step of admixing from about 5% (w/w) to about 10% (w/w) concentrated berry preparation with the bioadhesive carrier.

37. The method of claim 30, including the step of selecting a bioadhesive carrier which is a mucoadhesive gel.

38. The method of claim 37, including providing the mucoadhesive gel at a viscosity of from about 10,000 cP to about 20,000 cP.

39. A method for increasing the concentration of an anthocyanin in a bodily tissue or fluid of an individual at risk of an oral cancer or precancerous condition, comprising applying an isolated berry preparation containing said anthocyanin to an interior of an oral cavity of the individual.

40. The method of claim 39, wherein the bodily tissue or fluid is selected from the group consisting of a mucosal tissue, an oral mucosa tissue, oral tissue, peripheral blood, serum, and saliva.

41. The method of claim 39, wherein the isolated berry preparation is derived from the group consisting of strawberry, raspberry, red raspberry, black raspberry, and combinations thereof.

42. The method of claim 39, wherein the isolated berry preparation is derived by the steps of: (a) physically disrupting a quantity of berries; and (b) concentrating the physically disrupted berries by at least one of freeze-drying and solvent extraction.

43. The method of claim 42, including the step of admixing the isolated berry preparation with a mucoadhesive gel prior to applying to the oral cavity of the individual.

44. The method of claim 43, including the step of admixing from about 1% (w/w) to about 10% (w/w) concentrated berry preparation with the mucoadhesive gel.

45. The method of claim 44, including the step of admixing from about 5% (w/w) to about 10% (w/w) concentrated berry preparation with the mucoadhesive gel.

46. The method of claim 45, including the step of adjusting a pH of the mucoadhesive gel containing the isolated berry preparation to from about 6.0 to about 7.0 prior to applying the gel to the oral cavity of the individual.

47. The method of claim 46, wherein the pH of the mucoadhesive gel containing the isolated berry preparation is adjusted to about 6.5.

48. A method for improving the efficacy of a composition for chemoprevention of an oral cancer or precancerous condition, comprising:
providing an isolated berry preparation;
storing the isolated berry preparation at a first pH;
admixing the isolated berry preparation with a bioadhesive carrier to form a mixture; and
adjusting the mixture to a second pH which is different from the first pH prior to applying the mixture to an oral cavity of an individual in need of chemoprevention.

49. The method of claim 48, wherein the second pH is greater than the first pH.

50. The method of claim 49, wherein the first pH is from about 3.0 to about 4.5 and the second pH is from about 6.0 to about 7.0.

51. The method of claim 50, wherein the first pH is about 3.5 and the second pH is about 6.5.

52. The method of claim 48, including the step of deriving the isolated berry preparation from the group consisting of strawberry, raspberry, red raspberry, black raspberry, and combinations thereof.

53. The method of claim 52, wherein the isolated berry preparation is derived by the steps of physically disrupting a quantity of berries and concentrating the physically disrupted berries by at least one of freeze-drying or solvent extraction.

54. The method of claim 53, including the step of admixing from about 1% (w/w) to about 10% (w/w) concentrated berry preparation with the bioadhesive carrier.

55. The method of claim 54, including the step of admixing from about 5% (w/w) to about 10% (w/w) concentrated berry preparation with the bioadhesive carrier.

56. The method of claim 48, including selecting a bioadhesive carrier which is a mucoadhesive gel.

57. The method of claim 56, including providing the mucoadhesive gel at a viscosity of from about 10,000 cP to about 20,000 cP.
